(19) United States
(12) Patent Application Publication
(10) Pub. No.: US 2005/0090496 A1
(43) Pub. Date: Apr. 28, 2005

(54) Sulphonyl compounds with 5-ht6 receptor affinity

(76) Inventors: Mahmood Ahmed, Harlow (GB); Steven Mark Bromidge, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/503,682

(22) PCT Filed: Feb. 4, 2003
(86) PCT No.: PCT/EP03/01117

(30) Foreign Application Priority Data
Feb. 5, 2002 (GB) 0202679.7

(51) Int. Cl. A61K 31/519; A61K 31/503

(52) U.S. Cl. 514/248; 514/265.1

(57) ABSTRACT

The present invention relates to novel sulfonamide compounds having pharmacological activity, processes for their preparation, to compositions containing them and to their use in the treatment of CNS and other disorders.

This invention relates to novel sulphonyl compounds having pharmacological activity, processes for their preparation, to compositions containing them and to their use in the treatment of CNS and other disorders.

WO 98/27081, WO 99/02502, WO 99/37623, WO 99/42465 and WO 01/32646 (SmithKline Beecham plc) disclose a series of aryl sulphonamide and sulphoxide compounds as 5-Hr₆ receptor antagonists and which are claimed to be useful in the treatment of various CNS disorders. WO 01/39777 (Osi Pharmaceuticals) describes a series of substituted pyrrolo [2,3-b]pyrimidines as selective adenosine A1, A2a and A3 receptor antagonists. WO 99/65908 and WO 99/65909 (Pfizer) describe the use of 7H-pyrrolo [2,3A]pyrimidine-5-bromo-7-(phenylsulfonyl)-4-(1-piperidinyl) and 7H-pyrrolo [2,3-d]pyrimidine-5-iodo-7-(phenylsulfonyl)-4-(1-piperidinyl) as intermediates in the preparation of protein tyrosine kinases such as Janus Kinase 3. WO 99/28313 (Merck) describe a series of 1,2,3,4-tetrahydroisoquinolines and homologous compounds as farnesyl protein-transferases for chemotherapeutic applications.

A structurally novel class of compounds has now been found which also possess 5-HT₆ receptor affinity. The present invention therefore provides, in a first aspect, use of a compound of formula (I) or a pharmaceutically acceptable salt or solvate thereof: 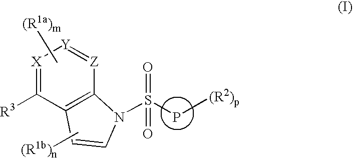
wherein:
P is aryl or heteroaryl;
R^{1a} and R^{1b} independently represent halogen, C₁₋₆ alkyl, C₁₋₆ alkoxy, C₁₋₆ alkanoyl, CN, CF₃, O CF₃, phenyloxy, benryloxy or C₃₋₆ cycloallyloxy;
R² is halogen, C₁₋₆ alkyl, C₃₋₆ cycloalkyl, C₁₋₆ alkoxy, C₁₋₆ alkylthio, C₁₋₆ alkylsulphinyl, C₁₋₆ alkylsulphonoyl, C₁₋₆ alkanoyl, CN, CF₃, OCH₂CF₃, OCF₃, hydroxy, hydroxyC₁₋₆ alkyl, hydroxyC₁₋₆ alkoxy, C₁₋₆ alkoxcarbonyl, C₁₋₁₆ alkoxyC₁₋₆ alkoxy, nitro, amino, N(C₁₋₆ alkyl)₂, NHC₁₋₆ alkyl, C₁₋₆ alkylamino or diC₁₋₆ alkylamino, C(O)OR⁴ (where R⁴ is hydrogen or C₁₋₆ alkyl), CONR⁵R⁶ or NR⁵COR⁶, (where R⁴ and R⁶ are independently hydrogen, Clot alkyl or R⁵ and R⁶ combine together to form a 5- to 7-membered azacyclic ring optionally containing an additional heteroatom selected from nitrogen, sulphur or oxygen), or aryl or heteroaryl (both of which may be optionally substituted by groups as defined for R^{1a} and R^{1b} above);
R³ is a 5- to 7-membered heterocyclic ring or a bicyclic heterocyclic ring containing 1 to 3 heteroatoms selected from nitrogen, sulphur or oxygen, said ring being optionally C- and/or N-substituted by one or more C₁₋₆ alkyl groups;
m and n independently represent an integer from 0-4;
p represents an integer from 0-5;
X, Y and Z independently represent nitrogen or carbon, provided that one or two of X, Y and Z represent nitrogen;
in the manufacture of a medicament for the treatment or prophylaxis of depression, anxiety, Alzheimers disease, age related cognitive decline, ADHD, obesity, mild cognitive impairment and schizophrenia.

As a second aspect of the present invention we provide a compound of formula (IA) or a pharmaceutically acceptable salt thereof which is a compound of formula (I) wherein R^{1a}, R^{1b}, R², R³, m, n, p, P, X, Y and Z are as defined above, with the proviso that the compound of formula (IA) is not
5-bromo-7(phenylsulfonyl)-4-(1-piperidinyl)-7H-pyrrolo [2,3-d]pyrimidine; or
5-iodo-7-(phenylsulfonyl)-4(1-piperidinyl)-7H-pyrrolo [2,3-d]pyrimidine.

Alkyl groups, whether alone or as part of another group, may be straight chain or branched and the groups alkoxy and alkanoyl shall be interpreted similarly. Alkyl moieties are more preferably C₁₋₄ alkyl, eg. methyl or ethyl. The term ‘halogen’ is used herein to describe, unless otherwise stated, a group selected from fluorine, chlorine, bromine or iodine.

The term “aryl” includes phenyl and naphthyl.

The term “heteroaryl” is intended to mean a 5 or 6 membered monocyclic aromatic or a fused 8-10 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such fused aromatic rings include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above.

It will be appreciated that wherein the above mentioned aryl or heteroaryl groups have more than one substituent, said substituents may be linked to form a ring, for example a carboxyl and amine group may be linked to form an amide group.

The term 5- to 7-membered heterocyclic ring is intended to mean a non aromatic ring containing 1 to 3 heteroatoms selected from oxygen, nitrogen and sulphur. Such rings may be partially unsaturated. Suitable examples of 5- to 7-membered heterocyclic rings include piperidinyl, tetrahydropyridinyl, pyrrolidinyl, morpholinyl, azepanyl, diazepanyl and piperazinyl. A 5- to 7-membered heterocyclic ring, as described above, may be linked to the remainder of the molecule via a carbon atom or a suitable nitrogen atom.

When R³ is a bicyclic heterocyclic ring, representative examples of such groups are:

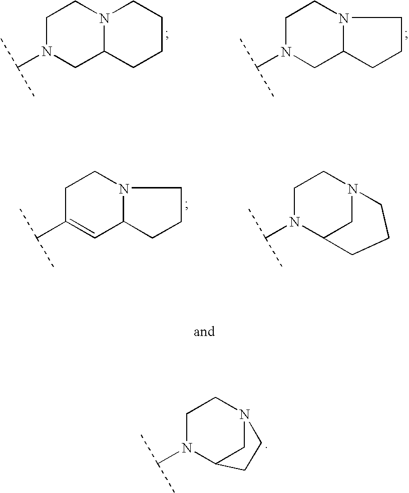

Preferably, P represents phenyl, pyridyl or pyrazolyl, more preferably phenyl.

Preferably, m represents 0.

Preferably, n represents 0.

Preferably, p represents 0 or 1, more preferably 1.

When present, R^{1a} and R^{1b} preferably independently represent halogen, a C₁₋₆alkyl group, CF₃, CN or a C₁₋₆alkoxy group.

Preferably, R² represents halogen, C₁₋₆ alkyl, trifluoromethoxy or trifluoromethyl, more preferably halogen.

Preferably, R³ has up to 2 substituents.

Preferably, R³ represents piperazinyl, more preferably unsubstituted piperazinyl.

Preferably, X and Y both represent carbon and Z represents nitrogen.

Preferred compounds according to the invention include example E1 as shown below, or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds of formula (I) may be prepared in crystalline or noncrystalline form, and, if crystalline, may optionally be solvated, eg. as the hydrate. This invention includes within its scope stoichiometric solvates (eg. hydrates) as well as compounds containing variable amounts of solvent (eg. water.).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (IA) or a pharmaceutically acceptable salt thereof, which process comprises:
(a) reacting a compound of formula (II) 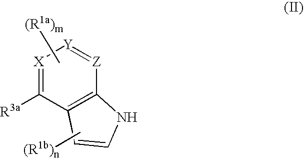
   wherein R¹, m, X, Y and Z are as defined above and R^{3a} represents an R³ group as defined above optionally protected with a suitable protecting group, eg. t-butoxycarbonyl (Boc), with a compound of formula (III) 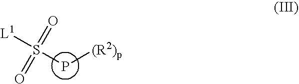
   wherein P, R² and n are as defined above and L¹ represents a suitable leaving group such as a halogen atom (eg. fluorine or chlorine); and as necessary, deprotecting a compound of formula (IA) which is protected;
(b) deprotecting a compound of formula (IA) which is protected; or
(c) interconversion to other compounds of formula (IA);
   and thereafter optionally forming a pharmaceutically acceptable salt.

Process (a) typically comprises the use of a suitable base, eg. potassium t-butoxide in a suitable solvent, eg. tetrahydrofuran.

In process (b), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric or trifluoroacetic acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellran linker), which may be removed by acid catalysed hydrolysis, for example with trifluoroacetic acid.

Process (c) may be performed using conventional interconversion procedures such as epimerisation, oxidation, reduction, alkylation, nucleophilic or electrophilic aromatic substitution, ester hydrolysis or amide bond formation.

Compounds of formula (II) wherein R³ represent piperazinyl may be prepared in accordance with the following process: 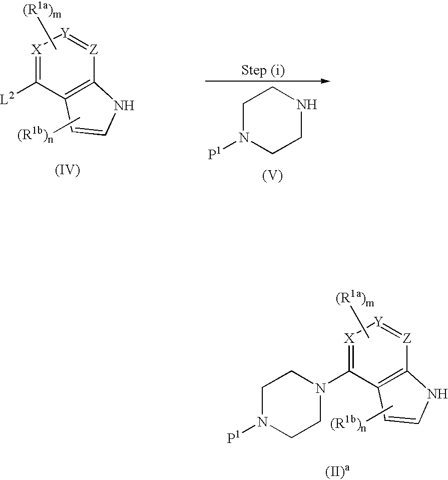
wherein R^{1a}, R^{1b}, m, n, X, Y and Z are as defined above, L² represents a suitable leaving group such as a halogen atom (eg. chlorine or fluorine), P¹ represents a suitable protecting group, such as t-butoxycarbonyl.

Step (i) typically comprises the use of a suitable solvent, eg. dimethylformamide in the presence of a suitable base, eg. using an excess of a compound of formula (V).

It will be appreciated that compounds of formula (II) wherein R³ represents N-linked groups other than piperazinyl may be prepared in an analogous manner to that described in the above process.

Compounds of formulae (II), (III), (IV) and (V) are commercially available, may be prepared using procedures described herein or by analogous methods thereto or according to known methods.

Pharmaceutically acceptable salts may be prepared conventionally by reaction with the appropriate acid or acid derivative.

Compounds of formula (I) and their pharmaceutically acceptable salts have affinity for the 5-HT₆ receptor and have potential use in the treatment of certain CNS disorders such as anxiety, depression, epilepsy, obsessive compulsive disorders, migraine, cognitive memory disorders (e.g. Alzheimers disease, age related cognitive decline and mild cognitive impairment), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. Compounds of the invention are also expected to be of use in the treatment of certain GI (gastrointestinal) disorderssuch as EBS (Irritable Bowel Syndrome).

Thus the invention also provides a compound of formula (I) or a pharmaceutically acceptable salt thereof, for use as a therapeutic substance, in particular in the treatment or prophylaxis of the above disorders. In particular the invention provides for a compound of formula (I) or a pharmaceutically acceptable salt thereof, for use in the treatment of depression, anxiety and cognitive memory disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment or prophylaxis of the above disorders.

In order to use the compounds of formula (I) in therapy, they will normally be formulated into a pharmaceutical composition in accordance with standard pharmaceutical practice.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

Description 1

### 4-(1H-Pyrrolo[2,3-b]pyridin-4-yl)piperazine-1-carboxylic Acid tert-butyl Ester (D1)

A mixture of 4-chloro-1H-pyrrolo[2,3-b]pyridine (100 mg, 0.66 mmol) [see Clark et al. J. Chem. Soc. Perlin Trans. 1 1974, 2270 for preparation] and piperazine-1-carboxylic acid tert-butyl ester (600 mg, 3.22 mmol) in DMF was heated to 150° C. for 4 h. After allowing to cool to room temperature, DCM was added and the organic phase then washed with water, brine, dried and concentrated in vacuo. Purification by column chromatography gave the title compound (D1) (70 mg) as a clear paste; δH (CDCl₃)/ppm 1.50 (9H, s), 3.47 (4H, t, J=4.9 Hz), 3.66 (4H, t, J=5.0 Hz), 6.43 (1H, d, J=5.6 Hz), 6.48 (1H, d, J=3.6 Hz), 7.18 (1H, d, J=3.6 Hz), 8.11 (1H, d, J=5.5 Hz), 9.40 (1H, br s); MS: m/z (M−H⁻) 301.

Description 2

### 4-[1-(3-Chlorobenzenesulfonyl)-1H-pyrrolo[2,3-b]pyridin-4yl]-piperazine-1-carboxylic Acid tert-butyl Ester (D2)

To an ice-cooled solution of 4-(1H-pyrrolo[2,3-b]pyridin-4-yl)-piperazine-1-carboxylic acid tert-butyl ester (D1) (40 mg, 0.13 mmol) in THF (3 mL) was added dropwise t-BuOK (0.15 mL, 0.15 mmol, 1.0 M in THF). After stirring for 20 min at this temperature, a solution of 3-chlorobenzenesulfonyl chloride (33 mg, 0.16 mmol) in TBF (2 mL) was added dropwise and the mixture allowed to warm to room temperature. Water was added after 3 h and the mixture extracted with diethyl ether and the organic phase dried. Evaporation of solvent was followed by purification by column chromatography to give the title compound (D2) as an orange gum (30 mg); δH (CDCl₃)/ppm 1.48 (9H, s), 3.37 (4H, t, J=5.0 Hz), 3.61 (4H, t, J=5.1 Hz), 6.51 (1H, d, J=5.6 Hz), 6.60 (1H, d, J=4.1 Hz), 7.41 (1H, t, J=8.0 Hz), 7.52 (1H, m), 7.57 (1H, d, J=4.0 Hz), 8.09 (1H, m), 8.19 (1H, t, J=0.5 Hz), 8.21 (1H, t, J=5.7 Hz); MS: m/z (M+H⁺) 477.

### EXAMPLE 1

### 4[1-(3-Chlorobenzenesulfonyl)-1-pyrrolo[2,3-b]pyridin-4-yl]-piperazine hydrochloride (E1)

4-[1-(3-Chorobenzenesulfonyl)-1H-pyrrolo[2,3-b]pyridin-4-yl]-piperazine-1-carboxylic acid tert-butyl ester (D2) (25 mg, 0.05 mmol) was exposed to 20% TFA in DCM for 1 h. Evaporation in vacuo, treatment with 1M HCl in diethyl ether in the presence of methanol and evaporation in vacuo gave the title compound (E1) as a clear paste (19 mg); δH (CD₃OD)/ppm 3.48 (4H, t, J=4.3 Hz), 4.16 (4H, t, J=4.7 Hz), 7.13 (1H, d, J=7.2 Hz), 7.24 (1H, d, J=4.2 Hz), 7.67 (1H, t, J=8.0 Hz), 7.83 (1H, m), 7.91 (1H, d, J=4.0 Hz), 8.11 (1H, m), 8.16 (1H, d, J=7.1 Hz), 8.26 (1H, m); MS: m/z (M+H⁺) 377.

Pharmacolorical Data

Compounds can be tested following the procedures outlined in WO98/27081.

The compound of Example E1 was tested and showed good affinity for the 5-HT₆ receptor, having pKi values >8 at human cloned 5-HT₆ receptors.

| Abbreviations | |
|---|---|
| TFA | trifluoroacetic acid |
| DCM | dichloromethane |
| DMF | dimethylformamide |
| THF | tetrahydrofuran |

1. A compound of formula (I) or a pharmaceutically acceptable salt or solvate thereof: 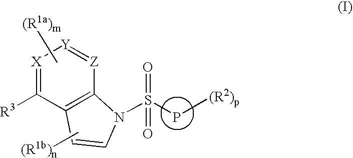
wherein:
R¹ represents hydrogen, halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, arylcarboxamidoC₁₋₆ alkyl, aroyl, aroylC₁₋₆ alkyl, arylC₁₋₆ alkanoyl, or a group CONR³R⁴ or SO₂NR³R⁴, wherein R³ and R⁴ independently represent hydrogen or C₁₋₆ alkyl or together may be fused to form a 5- to 7-membered aromatic or non-aromatic heterocyclic ring optionally interrupted by an O or S atom;
R² represents hydrogen or C₁₋₆ alkyl;
m represents an integer from 1 to 3;
n represents an integer from 1 to 4;
A represents phenyl, naphthyl or a monocyclic or bicyclic heteroaryl group each of which may be optionally substituted by one or more substituents which may be the same or different, and which are selected from those defined for R¹;
or solvates thereof.

2. A compound of formula (I) as defined in claim 1 which is
2,3,4,5-Tetrahydro-7-(3-trifluoromethyl)phenylsulfonamido-1H-benzo[d]azepine;
7-Phenylsulfonamido-2,3,4,5-tetrahydro-1H-benzo[d]azepine;
2,3,4,5-Tetrahydro-7-(3-chloro)phenylsulfonamido-1H-benzo[d]azepine;
2,3,4,5-Tetrahydro-7-(5-bromo-2-thienyl)sulfonamido-1H-benzo[d]azepine;
2,3,4,5-Tetrahydro-7-(4-methyl)phenylsulfonamido-1H-benzo[d]azepine;
7-(4-Bromo-2-trifluoromethoxy)phenylsulfonamido-2,3,4,5-tetrahydro-1H-benzo[d]azepine;
2,3,4,5-Tetrahydro-7-(2,3-dichloro)phenylsulfonamido-1H-benzo[d]azepine;
2,3,4,5-Tetrahydro-7-(3,5-dichloro-2-methoxy)phenylsulfonamido-1H-benzo[d]azepine;
2,3,4,5-Tetrahydro-7-(4-bromo-2-ethyl)phenylsulfonamido-1H-benzo[d]azepine;
7-(4-Chloro-2-trifluoromethoxy)phenylsulfonamido-2,3,4,5-tetrahydro-1H-benzo[d]azepine;
7-(Benzenesulfonylamino)-9-chloro-1,2,4,5-tetrahydro-benzo[d]azepine;
7-(3-trifluoromethyl-benzenesulfonylamino)-9-chloro-1,2,4,5-tetrahydro-benzo[d]azepine;
7-(Benzenesulfonylamino)-9-bromo-1,2,4,5-tetrahydro-benzo[d]azepine;
7-(4-Bromo-2-trifluoromethoxy-benzenesulfonylamino)-6-chloro-1,2,4,5-tetrahydro-benzo[d]azepine;
3,5-Dichloro-2-methoxy-N-(8-methoxy-2,3,4,5-tetrahydro-1H-benzo[d]azepin-7-yl)-benzenesulfonamide;
or a pharmaceutically acceptable salt thereof.

3. A pharmaceutical composition which comprises a compound according to claim 1 and a pharmaceutically acceptable carrier or excipient.

4. A method of treating depression, anxiety, Alzheimers disease, age related cognitive decline, ADHD, obesity, mild cognitive impairment and schizophrenia which comprises administering a safe and therapeutically effective amount to a patient in need thereof of a compound as defined in claim 1 or a pharmaceutically acceptable salt thereof.

5-15. canceled.
